(19) United States
(12) Patent Application Publication
(10) Pub. No.: US 2024/0352341 A1
(43) Pub. Date: Oct. 24, 2024

(54) CATALYST AND PROCESS FOR THE DEHYDROGENATION OF ALKANES TO OLEFINS

(71) Applicant: Dow Global Technologies LLC, Midland, MI (US)

(72) Inventors: Daniela Ferrari, Rosharon, TX (US); Barry B. Fish, Lake Jackson, TX (US); Kevin Blann, Missouri City, TX (US); Glenn Pollefeyt, Wondelgem (BE); Cheng L. Chung, Houston, TX (US); Manish Sharma, Missouri City, TX (US); Alexey Kirilin, Terneuzen (NL); Adam Chojecki, East Flanders (BE); Andrzej Malek, Midland, MI (US)

(73) Assignee: Dow Global Technologies LLC, Midland, MI (US)

(21) Appl. No.: 18/683,043

(22) PCT Filed: Aug. 17, 2022
(86) PCT No.: PCT/US2022/075067

§ 371 Date: Feb. 12, 2024

Related U.S. Application Data
(60) Provisional application No. 63/236,003, filed on Aug. 23, 2021.

(51) Int. Cl. C10G 27/04

(52) U.S. Cl. CPC C10G 27/04; C10G 2300/1081; C10G 2300/4006; C10G 2300/4012; C10G 2300/4018

(57) ABSTRACT

A method for converting alkanes to olefins includes contacting a feed stream comprising alkanes with an oxidative dehydrogenation that does not comprise tellurium catalyst in a reaction zone and dehydrogenating the alkanes without a co-feed of oxygen to yield a product stream having olefins. The oxidative dehydrogenation catalyst has the formula: MoᵥV_{w}Nb_{y}A_{z}Oₓ, where v is 1.0, w is from 0.1 to 0.5, y is from 0.001 to 0.3, A is Bi, Sb, Pr, or mixtures thereof, z is from 0.01 to 0.3, and x charge-balances the structure. The oxidative dehydrogenation catalyst has a crystallographic structure with Pba2-32 space group, characterized by reflections determined with Cu-K_{α} X-ray diffraction (XRD) as follows.

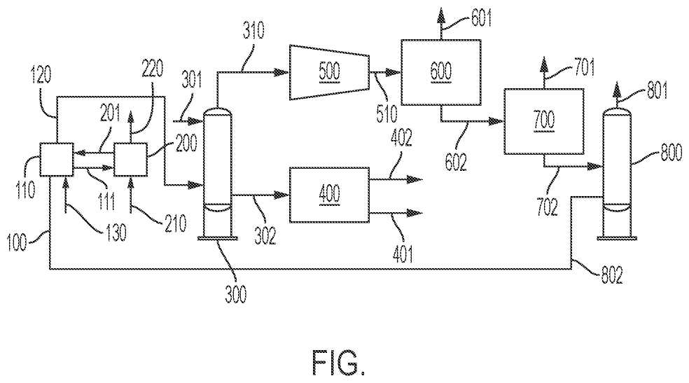

### CROSS-REFERENCE TO RELATED APPLICATIONS

The present application claims priority to U.S. Provisional Patent Application No. 63/236,003, filed Aug. 23, 2021, and entitled “CATALYST AND PROCESS FOR THE DEHYDROGENATION OF ALKANES TO OLEFINS,” the entirety of which is incorporated by reference herein.

### BACKGROUND

### Field

The present specification generally relates to catalysts and processes for the dehydrogenation of alkanes to olefins, such as catalysts and processes for converting ethane to ethylene.

### Technical Background

Conventional catalysts for converting alkanes to olefins, such as converting ethane to ethylene and acetic acid, are based on molybdenum (Mo), vanadium (V), and niobium (Nb) and include promoters such as calcium (Ca), sodium (Na), antimony (Sb), or tellurium (Te). In particular, Te is a common promoter included in the conventional catalysts. Processes using such catalysts require an oxygen co-feed and utilize an oxidative dehydrogenation process at low temperature, such as below 500° C., and low pressures, such as below 300 pounds per square inch gauge (psig) (about 20 barg).

### SUMMARY

According to one embodiment, a method for converting alkanes to olefins comprises: contacting a feed stream comprising alkanes with an oxidative dehydrogenation catalyst in a reaction zone, where the oxidative dehydrogenation catalyst does not comprise tellurium; and dehydrogenating the alkanes in the reaction zone without a co-feed of oxygen to yield a product stream comprising olefins, wherein the oxidative dehydrogenation catalyst has the following formula: MoᵥV_{w}Nb_{y}A_{z}Oₓ, where v is 1.0, w is from 0.1 to 0.5, y is from 0.001 to 0.3, A is Bi, Sb, Pr, or mixtures thereof, z is from 0.01 to 0.3, and x is an oxygen content required to charge-balance the structure, and the oxidative dehydrogenation catalyst has a crystallographic structure with Pba2-32 space group, characterized by reflections determined with Cu-K_{α} X-ray diffraction (XRD) as follows:

| 2θ (±0.3°) | Rel. Intensity (%) |
|---|---|
| 5.3 | 0.2-8 |
| 6.6 | 1.5-15 |
| 7.84 | 2.5-45 |
| 8.95 | 4-21 |
| 22.17 | 100 |
| 27.2 | 20-50 |
| 28.1 | 10-30. |

According to another embodiment, a method for converting alkanes to olefins comprises: contacting a feed stream comprising alkanes with an oxidative dehydrogenation catalyst in a reaction zone, where the oxidative dehydrogenation catalyst has the following formula: MoᵥV_{w}Nb_{y}Bi_{z}Oₓ, where v is 1.0, w is from 0.1 to 0.5, y is from 0.001 to 0.3, z is from 0.01 to 0.3, and x is an oxygen content required to charge-balance the structure, wherein the oxidative dehydration catalyst has a crystallographic structure with Pba2-32 space group, characterized by reflections determined with Cu-K_{α} X-ray diffraction (XRD) as follows:

| 2θ (±0.3°) | Rel. Intensity (%) |
|---|---|
| 5.3 | 0.2-8 |
| 6.6 | 1.5-15 |
| 7.84 | 2.5-45 |
| 8.95 | 4-21 |
| 22.17 | 100 |
| 27.2 | 20-50 |
| 28.1 | 10-30; |

and
dehydrogenating the alkanes in the reaction zone to yield a product stream comprising olefins.

Additional features and advantages will be set forth in the detailed description, which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### BRIEF DESCRIPTION OF THE DRAWING

The FIG. is a schematic drawing of a system for processing alkanes to olefins according to embodiments disclosed and described herein.

### DETAILED DESCRIPTION

Reference will now be made in detail to embodiments of processes for dehydrogenation of alkanes to olefins and of catalysts for the dehydrogenation of alkanes to olefins, such as processes and catalysts for converting ethane to ethylene.

One issue with conventional oxidative dehydrogenation processes is that they require a feed stream of oxygen gas (O₂). This adds costs to the process by requiring equipment that can produce pure, or nearly pure, oxygen to use in the process. In addition, the presence of oxygen in the process increases the chances for undesired, dangerous combustion as the oxygen and hydrocarbons mix. Finally, because of the nature of the catalyst, and the oxygen requirement for dehydrogenation of alkanes, conventional oxidative dehydrogenation processes for converting alkanes to olefins are conducted in fixed bed reactors, which require down time to remove, replace, and/or regenerate the catalyst. Accordingly, there is a need for improved catalyst that can convert alkanes to olefins.

It has unexpectedly been found that compositional modifications of the conventional oxidative dehydrogenation catalysts, as disclosed and described herein, allow for stable reduction and oxidation (redox) cycling of the materials. Catalysts disclosed and described herein have oxygen carrying capacity that is high enough so that selective conversion of ethane to ethylene is obtained in a circulating reactor fed with oxygenated solids. By using catalysts disclosed and described herein, circulation rates that are industrially viable in circulating reactors may be used and adequate conversion and selectivity of ethane to ethylene is achieved. This eliminates the need for feeding gas phase oxygen to the reactor. Moreover, air can be used to regenerate the spent catalyst. In addition, the reactor/regenerator system used for the ethane conversion is exothermic and, thus, can be operated without additional heat input.

Oxidative dehydrogenation catalysts comprising a crystalline structure of oxides of molybdenum, vanadium, niobium and one of bismuth, antimony or praseodymium disclosed and described herein may be used in processes for converting alkanes (also referred to herein as “paraffins”) in an alkane-containing feed stream to olefins. The processes disclosed and described herein may provide improved olefin selectivity by the oxidative dehydrogenation catalyst as time on stream increases. Processes disclosed and described herein generally include contacting a feed stream comprising alkanes (paraffins) with the oxidative dehydrogenation catalyst in a reaction zone, converting at least a portion of the alkanes to olefins yielding a product stream comprising paraffins and olefins. Finally, the paraffins and olefins in the product stream are separated, the paraffins may be recycled back to the feed stream, and the olefins are used in downstream systems or as materials in various products and processes. As the oxidative dehydrogenation catalyst in the reaction zone is utilized, its activity is reduced. According to embodiments, the used oxidative dehydrogenation catalyst will be removed from the reaction zone and sent to a regeneration zone where the catalyst will be regenerated by an oxygen-containing gas stream, such as air. Regenerated catalyst is then transferred from the regeneration zone back into the reaction zone, where it will be used to dehydrogenate alkanes in the feed stream to olefins. Processes according to embodiments disclosed and described herein will be provided in more detail below.

According to embodiments, and with reference to the FIG., a feed stream 100 is fed into a reaction zone 110, the feed stream 100 comprises at least one alkane. In embodiments, the feed stream may comprise steam and/or inert gas. In embodiments, the feed stream may be entirely comprised of alkanes (i.e., 100 vol % alkane). In one or more embodiments, the feed stream comprises from 30 volume percent (vol %) to 90 vol % alkane, from 35 vol % to 90 vol % alkane, from 40 vol % to 90 vol % alkane, from 45 vol % to 90 vol % alkane, from 50 vol % to 90 vol % alkane, from 55 vol % to 90 vol % alkane, from 60 vol % to 90 vol % alkane, from 65 vol % to 90 vol % alkane, from 70 vol % to 90 vol % alkane, from 75 vol % to 90 vol % alkane, from 80 vol % to 90 vol % alkane, from 85 vol % to 90 vol % alkane, from 30 vol % to 85 vol % alkane, from 35 vol % to 85 vol % alkane, from 40 vol % to 85 vol % alkane, from 45 vol % to 85 vol % alkane, from 50 vol % to 85 vol % alkane, from 55 vol % to 85 vol % alkane, from 60 vol % to 85 vol % alkane, from 65 vol % to 85 vol % alkane, from 70 vol % to 85 vol % alkane, from 75 vol % to 85 vol % alkane, from 80 vol % to 85 vol % alkane, from 30 vol % to 80 vol % alkane, from 35 vol % to 80 vol % alkane, from 40 vol % to 80 vol % alkane, from 45 vol % to 80 vol % alkane, from 50 vol % to 80 vol % alkane, from 55 vol % to 80 vol % alkane, from 60 vol % to 80 vol % alkane, from 65 vol % to 80 vol % alkane, from 70 vol % to 80 vol % alkane, from 75 vol % to 80 vol % alkane, from 30 vol % to 75 vol % alkane, from 35 vol % to 75 vol % alkane, from 40 vol % to 75 vol % alkane, from 45 vol % to 75 vol % alkane, from 50 vol % to 75 vol % alkane, from 55 vol % to 75 vol % alkane, from 60 vol % to 75 vol % alkane, from 65 vol % to 75 vol % alkane, from 70 vol % to 75 vol % alkane, from 30 vol % to 70 vol % alkane, from 35 vol % to 70 vol % alkane, from 40 vol % to 70 vol % alkane, from 45 vol % to 70 vol % alkane, from 50 vol % to 70 vol % alkane, from 55 vol % to 70 vol % alkane, from 60 vol % to 70 vol % alkane, from 65 vol % to 70 vol % alkane, from 30 vol % to 65 vol % alkane, from 35 vol % to 65 vol % alkane, from 40 vol % to 65 vol % alkane, from 45 vol % to 65 vol % alkane, from 50 vol % to 65 vol % alkane, from 55 vol % to 65 vol % alkane, from 60 vol % to 65 vol % alkane, from 30 vol % to 60 vol % alkane, from 35 vol % to 60 vol % alkane, from 40 vol % to 60 vol % alkane, from 45 vol % to 60 vol % alkane, from 50 vol % to 60 vol % alkane, from 55 vol % to 60 vol % alkane, from 30 vol % to 55 vol % alkane, from 35 vol % to 55 vol % alkane, from 40 vol % to 55 vol % alkane, from 45 vol % to 55 vol % alkane, from 50 vol % to 55 vol % alkane, from 30 vol % to 50 vol % alkane, from 35 vol % to 50 vol % alkane, from 40 vol % to 50 vol % alkane, from 45 vol % to 50 vol % alkane, from 30 vol % to 45 vol % alkane, from 35 vol % to 45 vol % alkane, from 40 vol % to 45 vol % alkane, from 30 vol % to 40 vol % alkane, from 35 vol % to 40 vol % alkane, or from 30 vol % to 35 vol % alkane. In embodiments, the at least one alkane is selected from the group consisting of ethane, propane, and combinations thereof. The inert gas is, in one or more embodiments selected from the group consisting of nitrogen, carbon dioxide and combinations thereof.

In embodiments, the feed stream is essentially free from oxygen, meaning that the feed stream comprises less than 2.0 volume percent (vol %) oxygen, less than 1.5 vol % oxygen, or less than 0.5 vol % oxygen. In one or more embodiments, the feed stream is free of oxygen.

The reaction zone is not particularly limited and any type of reactor allowing for cyclic or continuous operation of the process may be used in embodiments. The reaction zone is not particularly limited to a single reaction zone and can consist of multiple reactors in either series or parallel configuration. In one or more embodiments, the reaction zone may be a fluidized bed reactor, a moving bed reactor, a fixed bed reactor, a reverse flow reactor, or an ebullated bed reactor. The feed stream 100, which comprises alkanes, is fed into the reaction zone 110 and travels from a first end of the reaction zone 110 to a second end of the reaction zone 110 that is opposite to the first end of the reaction zone 110. As the feed stream 100 traverse from the first end of the reaction zone 110 to the second end of the reaction zone 110, the feed stream is contacted with the oxidative dehydrogenation catalyst that has been loaded into the reaction zone 110. Upon contact with the oxidative dehydrogenation catalyst—and at the proper reaction conditions which are described in more detail below—alkanes present in the feed stream 100 are converted to olefins. Accordingly, an effluent stream 120 that comprises alkanes and olefins exits the reaction zone 110.

According to one or more embodiments, the weight ratio of the oxidative dehydrogenation catalyst in the reaction zone 110 to alkane in the reaction zone 110 is from 250 to 10, from 225 to 10, from 200 to 10, from 175 to 10, from 150 to 10, from 125 to 10, from 100 to 10, from 75 to 10, from 50 to 10, from 25 to 10, from 250 to 25, from 225 to 25, from 200 to 25, from 175 to 25, from 150 to 25, from 125 to 25, from 100 to 25, from 75 to 25, from 50 to 25, from 250 to 50, from 225 to 50, from 200 to 50, from 175 to 50, from 150 to 50, from 125 to 50, from 100 to 50, from 75 to 50, from 250 to 75, from 225 to 75, from 200 to 75, from 175 to 75, from 150 to 75, from 125 to 75, from 100 to 75, from 250 to 100, from 225 to 100, from 200 to 100, from 175 to 100, from 150 to 100, from 125 to 100, from 250 to 125, from 225 to 125, from 200 to 125, from 175 to 125, from 150 to 125, from 250 to 150, from 225 to 150, from 200 to 150, from 175 to 150, from 250 to 175, from 225 to 175, from 200 to 175, from 250 to 200, from 225 to 200, or from 250 to 225. In embodiments where the reaction zone in a fluidized bed catalyst or the like, the catalyst to alkane ratio is controlled by the mass feed rate of alkane and the mass feed rate of catalyst to the reaction zone.

The feed stream 100 is contacted with the oxidative dehydrogenation catalyst as disclosed and described herein in the reaction zone 110 under reaction conditions sufficient to form a product stream 120 comprising olefins. The reaction conditions comprise a temperature within the reaction zone 110 that, according to one or more embodiments, is from 300° C. to 700° C., from 350° C. to 700° C., from 400° C. to 700° C., from 450° C. to 700° C., from 500° C. to 700° C., from 550° C. to 700° C., from 600° C. to 700° C., from 650° C. to 700° C., from 300° C. to 650° C., from 350° C. to 650° C., from 400° C. to 650° C., from 450° C. to 650° C., from 500° C. to 650° C., from 550° C. to 650° C., from 600° C. to 600° C., from 300° C. to 600° C., from 350° C. to 600° C., from 400° C. to 600° C., from 450° C. to 600° C., from 500° C. to 600° C., from 550° C. to 600° C., from 300° C. to 550° C., from 350° C. to 550° C., from 400° C. to 550° C., from 450° C. to 550° C., from 500° C. to 550° C., from 300° C. to 500° C., from 350° C. to 500° C., from 400° C. to 500° C., from 450° C. to 500° C., from 300° C. to 450° C., from 350° C. to 450° C., from 400° C. to 450° C., from 300° C. to 400° C., from 350° C. to 400° C., or from 300° C. to 350° C.

The reaction conditions also, in embodiments, include a pressure inside the reaction zone from 0 bar(g) (0 KPa) to 20 bar(g) (2000 KPa), from 5 bar(g) (500 KPa) to 20 bar(g) (2000 KPa), from 10 bar(g)(1000 KPa) to 20 bar(g) (2000 KPa), from 15 bar(g) (1500 KPa) to 20 bar(g)(2000 KPa), from 0 bar(g) (0 KPa) to 15 bar(g) (1500 KPa), form 5 bar(g) (500 KPa) to 15 bar(g) (1500 KPa), from 10 bar(g) (1000 KPa) to 15 bar(g) (1500 KPa), from 0 bar(g) (0 KPa) to 10 bar(g) (1000 KPa), form 5 bar(g)(500 KPa) to 10 bar(g)(1000 KPa), or from 0 bar(g)(0 KPa) to 5 bar(g) (500 KPa).

According to embodiments, the alkane weight hour space velocity (WHSV) of the feed stream 100 within the reaction zone 110 is from 0.1 per hour (/h) to 10/h, from 1/h to 10/h, from 2/h to 10/h, from 3/h to 10/h, from 4/h to 10/h, from 5/h to 10/h, from 6/h to 10/h, from 7/h to 10/h, from 8/h to 10/h, from 9/h to 10/h, from 1/h to 9/h, from 2/h to 9/h, from 3/h to 9/h, from 4/h to 9/h, from 5/h to 9/h, from 6/h to 9/h, from 7/h to 9/h, from 8/h to 9/h, from 1/h to 8/h, from 2/h to 8/h, from 3/h to 8/h, from 4/h to 8/h, from 5/h to 8/h, from 6/h to 8/h, from 7/h to 8/h, from 1/h to 7/h, from 2/h to 7/h, from 3/h to 7/h, from 4/h to 7/h, from 5/h to 7/h, from 6/h to 7/h, from 1/h to 6/h, from 2/h to 6/h, from 3/h to 6/h, from 4/h to 6/h, from 5/h to 6/h, from 1/h to 5/h, from 2/h to 5/h, from 3/h to 5/h, from 4/h to 5/h, from 1/h to 4/h, from 2/h to 4/h, from 3/h to 4/h, from 1/h to 3/h, from 2/h to 3/h, or from 1/h to 2/h.

According to embodiments, the reaction zone 110 may be fluidly connected to a regeneration zone 200 via a conduit 111. The configuration of the conduit 111 is not particularly limited provided that the conduit 111 is capable of transferring used oxidative dehydrogenation catalyst from the reaction zone 110 to the regeneration zone 200. In one or more embodiments, the regeneration zone 200 may be physically integrated with the reaction zone and may, in embodiments, be activated by providing an alternative feed gas (such as providing air in place of a hydrocarbon or alkane feed). At the regeneration zone 200, the used oxidative dehydrogenation catalyst is regenerated by contacting the used oxidative dehydrogenation catalyst with an oxygen-containing gas stream 210. In embodiments, the oxygen-containing gas stream 210 is air. As the oxidative dehydrogenation catalyst traverses from a first end of the regeneration zone 200 toward a second end of the regeneration zone 200, the residence time with the oxygen-containing gas stream 210 regenerates the oxidative dehydrogenation catalyst so that it regains its activity and selectivity for converting alkanes to olefins. After the oxidative dehydrogenation catalyst has be regenerated in the regeneration zone 200, the regenerated oxidative dehydrogenation catalyst is transferred from the regeneration zone 200 to the reaction zone 110 via a conduit 201. The configuration of the conduit 201 is not limited provided it allows the transfer of the regenerated oxidative dehydrogenation catalyst from the regeneration zone 200 to the reaction zone 100. It should be understood that fresh catalyst can be introduced into the reaction zone 110 via a different conduit (not shown) than the conduit 201 for introducing regenerated catalyst into the reaction zone 110. An effluent 220 exits the second end of the regeneration zone 200. In embodiments, the effluent 220 is nitrogen or oxygen-deprived air.

In embodiments, the oxygen-containing gas stream may comprise from 2 vol % to 22 vol % O₂, from 5 vol % to 22 vol % O₂, from 7 vol % to 22 vol % O₂, from 10 vol % to 22 vol % O₂, from 12 vol % to 22 vol % O₂, from 15 vol % to 22 vol % O₂, from 17 vol % to 22 vol % O₂, from 20 vol % to 22 vol % O₂, from 2 vol % to 20 vol % O₂, from 5 vol % to 20 vol % O₂, from 7 vol % to 20 vol % O₂, from 10 vol % to 20 vol % O₂, from 12 vol % to 20 vol % O₂, from 15 vol % to 20 vol % O₂, from 17 vol % to 20 vol % O₂, from 2 vol % to 17 vol % O₂, from 5 vol % to 17 vol % O₂, from 7 vol % to 17 vol % O₂, from 10 vol % to 17 vol % O₂, from 12 vol % to 17 vol % O₂, from 15 vol % to 17 vol % O₂, from 2 vol % to 15 vol % O₂, from 5 vol % to 15 vol % O₂, from 7 vol % to 15 vol % O₂, from 10 vol % to 15 vol % O₂, from 12 vol % to 15 vol % O₂, from 2 vol % to 12 vol % O₂, from 5 vol % to 12 vol % O₂, from 7 vol % to 12 vol % O₂, from 10 vol % to 12 vol % O₂, from 2 vol % to 10 vol % O₂, from 5 vol % to 10 vol % O₂, from 7 vol % to 10 vol % O₂, from 2 vol % to 7 vol % O₂, from 5 vol % to 7 vol % O₂, or from 2 vol % to 5 vol % O₂. In embodiments, the oxygen-containing gas stream is diluted or undiluted air. In other embodiments, the oxygen-containing stream can have an oxygen concentration greater than air, such as an oxygen concentration greater than 50%, greater than 70%, or greater than 90%.

According to embodiments the pressure in the regeneration zone 200 during the regeneration is from 0 bar(g) (0 KPa) to 21 bar(g) (2100 KPa), 2 bar(g) (200 KPa) to 21 bar(g) (2100 KPa), 4 bar(g) (400 KPa) to 21 bar(g) (2100 KPa), 6 bar(g) (600 KPa) to 21 bar(g) (2100 KPa), 8 bar(g) (800 KPa) to 21 bar(g) (2100 KPa), 10 bar(g) (1000 KPa) to 21 bar(g) (2100 KPa), 12 bar(g) (1200 KPa) to 21 bar(g) (2100 KPa), 14 bar(g) (1400 KPa) to 21 bar(g) (2100 KPa), 16 bar(g) (1600 KPa) to 21 bar(g) (2100 KPa), 18 bar(g) (1800 KPa) to 21 bar(g) (2100 KPa), 20 bar(g) (2000 KPa) to 21 bar(g) (2100 KPa), 0 bar(g) (0 KPa) to 20 bar(g) (2000 KPa), 2 bar(g) (200 KPa) to 20 bar(g) (2000 KPa), 4 bar(g) (400 KPa) to 20 bar(g) (2000 KPa), 6 bar(g) (600 KPa) to 20 bar(g) (2000 KPa), 8 bar(g) (800 KPa) to 20 bar(g) (2000 KPa), 10 bar(g) (1000 KPa) to 20 bar(g) (2000 KPa), 12 bar(g) (1200 KPa) to 20 bar(g) (2000 KPa), 14 bar(g) (1400 KPa) to 20 bar(g) (2000 KPa), 16 bar(g) (1600 KPa) to 20 bar(g) (2000 KPa), 18 bar(g) (1800 KPa) to 20 bar(g) (2000 KPa), 0 bar(g) (0 KPa) to 14 bar(g) (1400 KPa), 2 bar(g) (140 KPa) to 14 bar(g) (1400 KPa), 4 bar(g) (400 KPa) to 14 bar(g) (1400 KPa), 6 bar(g) (600 KPa) to 14 bar(g) (1400 KPa), 8 bar(g) (800 KPa) to 14 bar(g) (1400 KPa), 10 bar(g) (1000 KPa) to 14 bar(g) (1400 KPa), 12 bar(g) (1200 KPa) to 14 bar(g) (1400 KPa), 0 bar(g) (0 KPa) to 12 bar(g) (1200 KPa), 2 bar(g) (120 KPa) to 12 bar(g) (1200 KPa), 4 bar(g) (400 KPa) to 12 bar(g) (1200 KPa), 6 bar(g) (600 KPa) to 12 bar(g) (1200 KPa), 8 bar(g) (800 KPa) to 12 bar(g) (1200 KPa), 10 bar(g) (1000 KPa) to 12 bar(g) (1200 KPa), 0 bar(g) (0 KPa) to 10 bar(g) (1000 KPa), 2 bar(g) (100 KPa) to 10 bar(g) (1000 KPa), 4 bar(g) (400 KPa) to 10 bar(g) (1000 KPa), 6 bar(g) (600 KPa) to 10 bar(g) (1000 KPa), 8 bar(g) (800 KPa) to 10 bar(g) (1000 KPa), 0 bar(g) (0 KPa) to 8 bar(g) (800 KPa), 2 bar(g) (80 KPa) to 8 bar(g) (800 KPa), 4 bar(g) (400 KPa) to 8 bar(g) (800 KPa), 6 bar(g) (600 KPa) to 8 bar(g) (800 KPa), 0 bar(g) (0 KPa) to 6 bar(g) (600 KPa), 2 bar(g) (60 KPa) to 6 bar(g) (600 KPa), 4 bar(g) (400 KPa) to 6 bar(g) (600 KPa), 0 bar(g) (0 KPa) to 4 bar(g) (400 KPa), 2 bar(g) (40 KPa) to 4 bar(g) (400 KPa), or 0 bar(g) (0 KPa) to 2 bar(g) (200 KPa).

In embodiments, product stream 120 comprises various oxygenates in combination with alkanes and olefins. Accordingly, in embodiments product stream 120 is transferred from the reaction zone 110 to an oxygenates scrubber 300, where oxygenates are removed from the product stream 120. The oxygenates scrubber 300 may be any conventional oxygenates scrubber and is not limited herein. Product stream 120 enters a first end of the oxygenates scrubber 300 and travels to a second end of the oxygenates scrubber 300, and a water stream 301 is added to the oxygenates scrubber 300 near the second end of the oxygenates scrubber 300. As the product stream 120 traverses from the first end of the oxygenates scrubber 300 to the second end of the oxygenates scrubber 300, oxygenates are removed from the product stream 120. An oxygenate stream 302 exits the oxygenates scrubber 300 near the first end of the oxygenates scrubber 300.

The oxygenate stream 302 is then transferred from the oxygenates scrubber 300 to an oxygenates refiner 400 where oxygenates and water present in the oxygenates stream 302 are separated. The oxygenates refiner 400 may be any conventional oxygenates refiner and is not limited herein. Oxygenate stream 302 enters a first end of the oxygenates refiner 400 and travels to a second end of the oxygenates refiner 400. As the oxygenates stream 302 traverses from the first end of the oxygenates refiner 400 to the second end of the oxygenates refiner 400, oxygenates are separated from water in the oxygenates stream 302. An oxygenate stream 401 and a water stream 402 exit the oxygenates refiner 400 at the second end of the oxygenates refiner 400.

A refined product stream 310 exits the second end of the oxygenates scrubber 300. The refined product stream 310 comprises significantly less oxygenates than product stream 120 that exited the reaction zone 110. However, refined product stream 310 comprises carbon monoxide (CO) and carbon dioxide (CO₂) in addition to alkanes and olefins. Accordingly, refined product stream 310 is further processed by being transferred to a compressor where the refined product stream 310 is compressed. The compressor 500 may be any conventional compressor and is not limited herein. Once compressed, the compressed, refined product stream 510 is transferred to a CO₂ separator 600.

At the CO₂ separator 600, CO₂ is separated from CO, alkanes, and olefins in the compressed, refined product stream 510. The CO₂ separator may be any conventional CO₂ separator and is not limited herein. Carbon dioxide 601 is purged from the CO₂ separator, and a separated product stream 602 exits the CO₂ separator for further processing. The separated product steam 602 comprises CO, alkanes, and olefins.

The separated product stream 602 is transferred to a CO separator 700. At the CO separator 700, CO is separated from alkanes and olefins in the separated product stream 602. The CO separator may be any conventional CO separator and is not limited herein. Carbon monoxide 701 is purged from the CO separator, and a further separated product stream 702 exits the CO separator for further processing. The further separated product steam 702 comprises alkanes and olefins.

The components of the further separated product stream 702 can be separated with conventional separation units, which may optionally be part of an existing cracker separation system. In embodiments, the further separated product stream 702 is transferred to an olefin/paraffin splitter, 800. At the splitter, 800, alkanes are separated from olefins in the further separated product stream 702. The splitter may be any conventional cracker and is not limited herein. A final product stream 801 comprising olefins, such as ethylene, exits a first end of the cracker 800 and an alkane recycle stream 802 exits the cracker 800 and is returned to the reaction zone 110.

Catalysts for dehydrogenating alkanes to olefins according to embodiments disclosed and described herein will now be described.

One currently used oxidative dehydrogenation catalyst comprises MoVNbTeOₓ. The crystal phase structure, or a similar crystal phase structure, of the catalyst formed by MoVNbTeOₓ (Pba2-32 space group) provides a structure that makes it possible to yield desired olefins. However, using this catalyst in an oxidative dehydrogenation process leads to significant catalyst instability because Te is volatile under reducing conditions, causing reactor contamination with Te as well as potential collapse of the preferred crystalline structure of the catalyst. This will subsequently lead to activity/selectivity loss during the alkane to olefin conversion.

In embodiments disclosed and described herein, Te can be completely replaced in the MoVNbTeOₓ catalyst composition with a promoter. In embodiments, the promoter is selected from the group consisting of bismuth (Bi), antimony (Sb), or praseodymium (Pr). In one or more embodiments, the promoter is bismuth (Bi). Further, by using a specific hydrothermal synthesis method, which is disclosed in more detail herein, the catalyst may have a crystal structure that is sufficiently similar to MoVNbTeOₓ such that the alkane to olefin conversion provides desired olefins. The oxidative dehydrogenation catalyst has a Pba2-32 space group crystal structure. This structure replaces the volatile Te with a more stable Bi, Sb, Pr or combinations thereof, which allows for improved stability over the known MoVNbTeOₓ catalysts while providing similar alkane conversion. For instance, in embodiments the oxidative dehydrogenation catalyst disclosed and described herein is both active (greater than 10% Ethane conversion), selective (greater than 65% ethylene selectivity), and renders stable performance under reaction conditions. In one or more embodiments, the catalysts described herein may be further promoted by sodium (Na) or calcium (Ca).

In one or more embodiments, the oxidative dehydrogenation catalyst has the following chemical formula: MoᵥV_{w}Nb_{y}A_{z}Oₓ, where v is 1.0 (e.g., Mo is used as the basis for the atomic ratios), w is from 0.1 to 0.5, y is from 0.001 to 0.3, A is Bi, Sb, Pr, or combinations thereof, z is from 0.01 to 0.3, and x is the oxygen content required to charge-balance the structure. In embodiments, w is from 0.1 to 0.5, from 0.2 to 0.5, from 0.3 to 0.5, from 0.4 to 0.5, from 0.1 to 0.4, from 0.2 to 0.4, from 0.3 to 0.4, from 0.1 to 0.3, from 0.2 to 0.3, or from 0.1 to 0.2. In embodiments, y is from 0.01 to 0.3, from 0.05 to 0.3, from 0.1 to 0.3, from 0.15 to 0.3, from 0.2 to 0.3, from 0.25 to 0.3, from 0.001 to 0.25, from 0.01 to 0.25, from 0.05 to 0.25, from 0.1 to 0.25, from 0.15 to 0.25, from 0.2 to 0.25, from 0.01 to 0.2, from 0.05 to 0.2, from 0.1 to 0.2, from 0.15 to 0.2, from 0.01 to 0.15, from 0.05 to 0.15, from 0.1 to 0.15, from 0.01 to 0.1, from 0.05 to 0.1, or from 0.01 to 0.05. In embodiments, z is from 0.05 to 0.3, from 0.10 to 0.3, from 0.15 to 0.3, from 0.2 to 0.3, from 0.25 to 0.3, from 0.01 to 0.25, is from 0.05 to 0.25, from 0.10 to 0.25, from 0.15 to 0.25, from 0.2 to 0.25, from 0.01 to 0.2, is from 0.05 to 0.2, from 0.10 to 0.2, from 0.15 to 0.2, from 0.01 to 0.15, is from 0.05 to 0.15, from 0.10 to 0.15, from 0.01 to 0.1, is from 0.05 to 0.1, or from 0.01 to 0.05. In embodiments, the oxidative dehydrogenation catalyst has the following formula: MoV_{0.2.-0.3}Nb_{0.1}A_{0.1}Oₓ, where x is the oxygen content required to charge-balance the structure and A is selected from the group consisting of Bi, Sb, Pr, or combinations thereof. In embodiments, A is one of Bi or Sb. It should be understood that embodiments of the MoᵥV_{w}Nb_{y}A_{z}Oₓ catalyst having a Pba2-32 space group is essentially free of Te, such as having a Te/Mo ratio below 0.01.

It has been found that the presence of Nb in the oxidative dehydrogenation catalyst having the structure MoᵥV_{w}Nb_{y}A_{z}Oₓ and a Pba2-32 space group crystal structure improves catalyst activity and selectivity in a lattice oxidative dehydrogenation process (which is where oxygen for the conversion is extracted from the lattice of the catalyst rather than through a gaseous oxygen stream). Accordingly, in embodiments, the oxidative dehydrogenation catalyst consists of a structure comprising oxides of Mo, V, Nb, and Bi having the formula MoᵥV_{w}Nb_{y}Bi_{z}Oₓ and a Pba2-32 space group crystal structure.

The crystal structure of the oxidative dehydrogenation catalyst disclosed and described herein can, in embodiments, also be measured using x-ray diffraction (XRD). For instance, and as would be understood by a skilled artisan, the relative intensity of XRD peaks at various angles can be used to describe the crystal structure of the oxidative dehydrogenation catalyst. In embodiments, the oxidative dehydrogenation catalyst has reflections determined with Cu-K_{α} XRD as shown in Table 1. In Table 1 below, the relative intensity (Rel. Intensity) is the largest when 2θ is 22.2° and, thus, this relative intensity is set to 100% and used as the basis for the remaining relative intensities shown in Table 1.

TABLE 1

| 2θ (±0.3°) | Rel. Intensity (%) |
|---|---|
| 5.3 | 0.2-8 |
| 6.6 | 1.5-15 |
| 7.84 | 2.5-45 |
| 8.95 | 4-21 |
| 22.17 | 100 |
| 27.2 | 20-50 |
| 28.1 | 10-30 |

As would be recognized by one of ordinary skill in the art, the relative intensity may be affected by the preferential orientation effect and the above-disclosed relative intensities take into account such effects.

When the catalyst comprising Bi, Pr or combinations thereof as disclosed above is used in the reaction zone, an oxygen stream 130 may, optionally, be added to the reaction zone 110. It should be understood that an oxygen stream 130 is not required, and embodiments disclosed and described herein do not include adding an oxygen stream 130 into the reaction zone 110. However, in embodiments, an oxygen stream 130 but can be added to facilitate reactions within the reaction zone 110. The concentration of oxygen in the oxygen stream 130 is not particularly limited. For example, the oxygen concentration in the oxygen stream 130 may be from 0.1 vol % to 99.9 vol %, such as from 5.0 vol % to 95.0 vol %, from 10.0 vol % to 90.0 vol %, from 15.0 vol % to 85.0 vol %, from 20.0 vol % to 80.0 vol %, from 25.0 vol % to 75.0 vol %, from 30.0 vol % to 70.0 vol %, from 35.0 vol % to 65.0 vol %, from 40.0 vol % to 60.0 vol %, or from 45.0 vol % to 55.0 vol %. In one or more embodiments, the concentration of oxygen in the oxygen stream is relatively low, such as from 0.1 vol % to 5.0 vol %, from 0.2 vol % to 5.0 vol %, from 0.5 vol % to 5.0 vol %, from 0.8 vol % to 5.0 vol %, from 1.0 vol % to 5.0 vol %, from 1.2 vol % to 5.0 vol %, from 1.5 vol % to 5.0 vol %, from 1.8 vol % to 5.0 vol %, from 2.0 vol % to 5.0 vol %, from 2.2 vol % to 5.0 vol %, from 2.5 vol % to 5.0 vol %, from 2.8 vol % to 5.0 vol %, from 3.0 vol % to 5.0 vol %, from 3.2 vol % to 5.0 vol %, from 3.5 vol % to 5.0 vol %, from 3.8 vol % to 5.0 vol %, from 4.0 vol % to 5.0 vol %, from 4.2 vol % to 5.0 vol %, from 4.5 vol % to 5.0 vol %, or from 4.8 vol % to 5.0 vol %.

The oxygen stream 130 may, in embodiments, be added to the reaction zone 110 sequentially to the feed stream 100, such that the feed stream 100 and the oxygen stream 130 are not added to the reaction zone 110 at the same time.

In one or more embodiments, the oxygen stream 130 is added to the reaction zone 110 simultaneously to the feed stream 100. In such embodiments, the volume ratio of oxygen (in the oxygen stream 130) to alkanes (in the feed stream 100) in the reaction zone 110 is from greater than 0.0 to 3.0, from 0.5 to 3.0, from 1.0 to 3.0, from 1.5 to 3.0, from 2.0 to 3.0, from 2.5 to 3.0, from greater than 0.0 to 2.5, from 0.5 to 2.5, from 1.0 to 2.5, from 1.5 to 2.5, from 2.0 to 2.5, from greater than 0.0 to 2.0, from 0.5 to 2.0, from 1.0 to 2.0, from 1.5 to 2.0, from greater than 0.0 to 1.5, from 0.5 to 1.5, from 1.0 to 1.5, from greater than 0.0 to 1.0, from 0.5 to 1.0, or from greater than 0.0 to 0.5.

As mentioned above, using a specific hydrothermal method for forming the oxidative dehydrogenation catalyst allows the oxidative dehydrogenation catalysts to be formed having the desired Pba2-32 crystal structure. Embodiments of these hydrothermal methods for forming the oxidative dehydrogenation catalyst will now be described in more detail.

Oxidative dehydrogenation catalysts having a MoᵥV_{w}Nb_{y}Bi_{z}Oₓ structure are, in one or more embodiments, formed through a synthetic process started by adding a molybdenum-containing compound, a vanadium-containing compound, a bismuth-containing compound, a niobium-containing compound, and one or more organic acids to a mixture of alkylene glycol or alcohol amines and water to form a reaction mixture. In embodiments, the metal precursors are chosen as such that the precursors can be dissolved/digested under hydrothermal reaction conditions MoᵥV_{w}Nb_{y}Bi_{z}Oₓ is then synthesized from the reaction mixture by hydrothermal synthesis at a hydrothermal synthesis temperature for a period of time. After the period of time has passed, MoᵥV_{W}Nb_{y}Bi_{z}Oₓ is separated from retained liquids. In one or more embodiments, the molybdenum-containing, vanadium-containing, bismuth-containing, niobium-containing compound, and one or more acids are added to the mixture of alkylene glycol and water sequentially.

In embodiments, the bismuth-containing compound is selected from the group consisting of bismuth oxide (Bi₂O₃), bismuth sulfate (Bi₂(SO₄)₃), bismuth citrate (BiC₆H₅O₇), and bismuth nitrate (Bi(NO₃)₃). In embodiments, the niobium-containing compound is selected from the group consisting of niobium oxide, niobic acid (Nb₂O₅·nH₂O), niobium ethoxide, and ammonium niobium oxalate and water ((NH₄)Nb(C₂O₄)₂·nH₂O). In embodiments, the molybdenum-containing compound can be ammonium heptamolybdate (NH₄)₆Mo₇O₂₄ or molybdenum trioxide (MoO₃), and the vanadium-containing compound can be ammonium metavanadate (NH₄VO₃), vanadyl sulfate (VOSO₄), or vanadium pentoxide (V₂O₅). The molybdenum-containing compound and the vanadium-containing compound are, in embodiments, MoO₃ and V₂O₅, respectively. In embodiments, the antimony-containing compound is selected from the group consisting of antimony oxide (Sb₂O₃ or Sb₂O₅), antimony sulfate (Sb₂(SO₄)₃) and antimony acetate ((CH₃CO₂)₃Sb). In one or more embodiments, the praseodymium-containing compound is selected from the group consisting of praseodymium oxide (PrO₂, Pr₂O₃ or Pr₆O₁₁), praseodymium sulfate (Pr₂(SO₄)₃) and praseodymium nitrate (Pr(NO₃)₃). In some embodiments, a digestible mixture of metal containing compounds having the correct stoichiometric ratio of one or more of Mo, V, Nb, and Bi could be used. Examples of such digestible mixtures include (Mo,V)Oₓ and BiNbOₓ. In one or more embodiments, the acid is selected from the group consisting of citric acid (C₆H₅O₇), oxalic acid (C₂H₂O₄), and mixtures thereof. In embodiments, the alkylene glycol is ethylene glycol.

The hydrothermal synthesis temperature is, in embodiments, from 150° C. to 250° C., from 160° C. to 250° C., from 170° C. to 250° C., from 180° C. to 250° C., from 190° C. to 250° C., from 200° C. to 250° C., from 210° C. to 250° C., from 220° C. to 250° C., from 230° C. to 250° C., from 240° C. to 250° C., from 150° C. to 240° C., from 160° C. to 240° C., from 170° C. to 240° C., from 180° C. to 240° C., from 190° C. to 240° C., from 200° C. to 240° C., from 210° C. to 240° C., from 220° C. to 240° C., from 230° C. to 240° C., from 150° C. to 230° C., from 160° C. to 230° C., from 170° C. to 230° C., from 180° C. to 230° C., from 190° C. to 230° C., from 200° C. to 230° C., from 210° C. to 230° C., from 220° C. to 230° C., from 150° C. to 220° C., from 160° C. to 220° C., from 170° C. to 220° C., from 180° C. to 220° C., from 190° C. to 220° C., from 200° C. to 220° C., from 210° C. to 220° C., from 150° C. to 210° C., from 160° C. to 210° C., from 170° C. to 210° C., from 180° C. to 210° C., from 190° C. to 210° C., from 200° C. to 210° C., from 150° C. to 200° C., from 160° C. to 200° C., from 170° C. to 200° C., from 180° C. to 200° C., from 190° C. to 200° C., from 150° C. to 190° C., from 160° C. to 190° C., from 170° C. to 190° C., from 180° C. to 190° C., from 150° C. to 180° C., from 160° C. to 180° C., from 170° C. to 180° C., from 150° C. to 170° C., from 160° C. to 170° C., or from 150° C. to 160° C.

In embodiments, the hydrothermal pressure is from 4 bar (400 kPa) to 40 bar (4000 kPa), such as from 5 bar (500 kPa) to 40 bar (4000 kPa), from 10 bar (1000 kPa) to 40 bar (4000 kPa), from 15 bar (1500 kPa) to 40 bar (4000 kPa), from 20 bar (2000 kPa) to 40 bar (4000 kPa), from 25 bar (2500 kPa) to 40 bar (4000 kPa), from 30 bar (3000 kPa) to 40 bar (4000 kPa), from 35 bar (3500 kPa) to 40 bar (4000 kPa), from 4 bar (400 kPa) to 35 bar (3500 kPa), from 5 bar (500 kPa) to 35 bar (3500 kPa), from 10 bar (1000 kPa) to 35 bar (3500 kPa), from 15 bar (1500 kPa) to 35 bar (3500 kPa), from 20 bar (2000 kPa) to 35 bar (3500 kPa), from 25 bar (2500 kPa) to 35 bar (3500 kPa), from 30 bar (3000 kPa) to 35 bar (3500 kPa), from 4 bar (400 kPa) to 30 bar (3000 kPa), from 5 bar (500 kPa) to 30 bar (3000 kPa), from 10 bar (1000 kPa) to 30 bar (3000 kPa), from 15 bar (1500 kPa) to 30 bar (3000 kPa), from 20 bar (2000 kPa) to 30 bar (3000 kPa), from 25 bar (2500 kPa) to 30 bar (3000 kPa), from 4 bar (400 kPa) to 25 bar (2500 kPa), from 5 bar (500 kPa) to 25 bar (2500 kPa), from 10 bar (1000 kPa) to 25 bar (2500 kPa), from 15 bar (1500 kPa) to 25 bar (2500 kPa), from 20 bar (2000 kPa) to 25 bar (2500 kPa), from 4 bar (400 kPa) to 20 bar (2000 kPa), from 5 bar (500 kPa) to 20 bar (2000 kPa), from 10 bar (1000 kPa) to 20 bar (2000 kPa), from 15 bar (1500 kPa) to 20 bar (2000 kPa), from 4 bar (400 kPa) to 15 bar (1500 kPa), from 5 bar (500 kPa) to 15 bar (1500 kPa), from 10 bar (1000 kPa) to 15 bar (1500 kPa), from 4 bar (400 kPa) to 10 bar (1000 kPa), or from 5 bar (500 kPa) to 10 bar (1000 kPa).

According to embodiments, after the MoᵥV_{w}Nb_{y}A_{z}Oₓ oxidative dehydrogenation catalyst is separated from the retained liquids, the MoᵥV_{w}Nb_{y}A_{z}Oₓ oxidative dehydrogenation catalyst is dried and optionally calcined by heating the dried MoᵥV_{w}Nb_{y}A_{z}Oₓ oxidative dehydrogenation catalyst to a calcination temperature and holding the MoᵥV_{w}Nb_{y}A_{z}Oₓ oxidative dehydrogenation catalyst at the calcination temperature for a period of time.

In embodiments, the calcination takes place in an inert atmosphere, such as nitrogen (N₂), argon (Ar), or helium (He). In such embodiments, the calcination temperature is from 350° C. to 650° C., from 375° C. to 650° C., 400° C. to 650° C., from 425° C. to 650° C., from 450° C. to 650° C., from 475° C. to 650° C., from 500° C. to 650° C., from 525° C. to 650° C., from 550° C. to 650° C., from 575° C. to 650° C., from 600° C. to 650° C., from 625° C. to 650° C., from 350° C. to 625° C., from 375° C. to 625° C., from 400° C. to 625° C., from 425° C. to 625° C., from 450° C. to 625° C., from 475° C. to 625° C., from 500° C. to 625° C., from 525° C. to 625° C., from 550° C. to 625° C., from 575° C. to 625° C., from 600° C. to 625° C., from 350° C. to 600° C., from 375° C. to 600° C., from 400° C. to 600° C., from 425° C. to 600° C., from 450° C. to 600° C., from 475° C. to 600° C., from 500° C. to 600° C., from 525° C. to 600° C., from 550° C. to 600° C., from 575° C. to 600° C., from 350° C. to 575° C., from 375° C. to 575° C., from 400° C. to 575° C., from 425° C. to 575° C., from 450° C. to 575° C., from 475° C. to 575° C., from 500° C. to 575° C., from 525° C. to 575° C., from 550° C. to 575° C., from 350° C. to 550° C., from 375° C. to 550° C., from 400° C. to 550° C., from 425° C. to 550° C., from 450° C. to 550° C., from 475° C. to 550° C., from 500° C. to 550° C., from 525° C. to 550° C., from 350° C. to 525° C., from 375° C. to 525° C., from 400° C. to 525° C., from 425° C. to 525° C., from 450° C. to 525° C., from 475° C. to 525° C., from 500° C. to 525° C., from 350° C. to 500° C., from 375° C. to 500° C., from 400° C. to 500° C., from 425° C. to 500° C., from 450° C. to 500° C., from 475° C. to 500° C., from 350° C. to 475° C., from 375° C. to 475° C., from 400° C. to 475° C., from 425° C. to 475° C., from 450° C. to 475° C., from 350° C. to 450° C., from 375° C. to 450° C., from 400° C. to 450° C., from 425° C. to 450° C., from 350° C. to 425° C., from 375° C. to 425° C., from 400° C. to 425° C. from 350° C. to 400° C., from 375° C. to 400° C., or from 350° C. to 375° C.

In embodiments, the calcination takes place in air. In such embodiments, the calcination temperature may be from 200° C. to 500° C., from 375° C. to 500° C., from 400° C. to 500° C., from 425° C. to 500° C., from 450° C. to 500° C., from 475° C. to 500° C., from 350° C. to 475° C., from 375° C. to 475° C., from 400° C. to 475° C., from 425° C. to 475° C., from 450° C. to 475° C., from 350° C. to 450° C., from 375° C. to 450° C., from 400° C. to 450° C., from 425° C. to 450° C., from 350° C. to 425° C., from 375° C. to 425° C., from 400° C. to 425° C. from 350° C. to 400° C., from 375° C. to 400° C., or from 350° C. to 375° C.

### EXAMPLES

### Example 1

A mixture of 34 mL of H₂O and 80 microliter of ethylene glycol was added to a 45 mL Teflon-insert autoclave (Model 4744 General Purpose Acid Digestion Vessel, Parr). While stirring the mixture, 2.7126 g of MoO₃, 0.5141 g of V₂O₅, 0.4373 g of Bi₂O₃, 0.286 g of Nb₂O₅·xH₂O, 0.2711 g of Citric acid and 0.2388 g of oxalic acid was added sequentially and stirred for 10 min. Hydrothermal synthesis of MoV_{0.3}Nb_{0.1}Bi_{0.1}Oₓ was performed in a rotating shaft oven at 180° C. for 48 hours rotating at 10 rpm. The obtained material from the hydrothermal synthesis was purified with 90 mL of deionized water using vacuum filtration and subsequently dried at 85° C. overnight.

After drying, the material was calcined at 450° C. (at a heating rate of 2° C./min) under a flow of N₂, for 2 hours. The material was compacted under 7 ton pressure and crushed and sieved to 40-80 mesh prior to loading in the reactor and tested at 1.25 bar(a) ethane pressure having a WHSV of 3.2/hr.

### Example 2

A mixture of 34 mL of H₂O and 160 microliter of ethylene glycol was added to a 45 mL Teflon-insert autoclave (Model 4744 General Purpose Acid Digestion Vessel, Parr). While stirring, 2.7126 g of MoO₃, 0.5141 g of V₂O₅, 0.4373 g of Bi₂O₃, 0.286 g of Nb₂O₅·xH₂O, 0.5422 g of Citric acid and 0.2388 g of oxalic acid was added sequentially and stirred for 10 min. Hydrothermal synthesis of MoV_{0.3}Nb_{0.1}Bi_{0.1}Oₓ was performed in a rotating shaft oven at 190° C. for 48 hours rotating at 10 rpm. The obtained material from the hydrothermal synthesis was purified with 90 mL of deionized water using vacuum filtration and subsequently dried at 85° C. overnight.

After drying, the material was calcined at 450° C. (at a heating rate of 2° C./min) under a flow of N₂, for 2 hours. The material was compacted under 7 ton pressure and crushed and sieved to 40-80 mesh prior to loading in the reactor and tested at 1.25 bar(a) (125 kPa) ethane pressure having a WHSV of 3.2/hr.

### Example 3

A mixture of 34 mL of H₂O and 80 microliter of ethylene glycol was added to a 45 mL Teflon-insert autoclave (Model 4744 General Purpose Acid Digestion Vessel, Parr). While stirring, 2.7126 g of MoO₃, 0.5141 g of V₂O₅, 0.4373 g of Bi₂O₃, 0.8416 g of (NH₄)Nb(C₂O4)₂·xH₂O and 0.2711 g of Citric acid was added sequentially and stirred for 10 min. Hydrothermal synthesis of MoV_{0.3}Nb_{0.1}Bi_{0.1}Oₓ was performed in a rotating shaft oven at 190° C. for 48 hours rotating at 10 rpm. The obtained material from the hydrothermal synthesis was purified using 90 mL of deionized water using vacuum filtration and subsequently dried at 85° C. overnight.

After drying, the material was calcined at 450° C. (at a heating rate of 2° C./min) under a flow of N₂, for 2 hours. The material was compacted under 7 ton pressure and crushed and sieved to 40-80 mesh prior to loading in the reactor and tested at 1.25 bar(a) (125 kPa) ethane pressure having a WHSV of 3.2/hr.

### Comparative Example 1

MoV_{0.3}Nb_{0.17}Te_{0.23}Oₓ was prepared according to the procedure described in U.S. Pat. No. 9,156,764 B2. The material was compacted under 7 ton pressure and crushed and sieved to 40-80 mesh prior to loading in the reactor and tested at 1.25 bar(a) (125 kPa) ethane pressure having a WHSV of 3.2/hr.

### Example 4

A mixture of 34 mL of H₂O and 80 microliter of ethylene glycol was added to a 45 mL Teflon-insert autoclave (Model 4744 General Purpose Acid Digestion Vessel, Parr). While stirring, 2.7126 g of MoO₃, 0.5141 g of V₂O₅, 0.3033 g of Sb₂O₅, 0.8416 g of (NH₄)Nb(C₂O₄)₂·xH₂O and 0.2711 g of Citric acid was added sequentially and stirred for 10 min. Hydrothermal synthesis of MoV_{0.3}Nb_{0.1}Sb_{0.1}Oₓ was performed in a rotating shaft oven at 190° C. for 48 hours rotating at 10 rpm. The obtained material from the hydrothermal synthesis was purified with 90 mL of deionized water using vacuum filtration and subsequently dried at 85° C. overnight.

After drying, the material was calcined at 450° C. (at a heating rate of 2° C./min) under a flow of N₂, for 2 hours. The material was compacted under 7 ton pressure and crushed and sieved to 40-80 mesh prior to loading in the reactor and tested at 1.25 bar(a) (125 kPa) ethane pressure having a WHSV of 3.2/hr.

### Example 5

A mixture of 34 mL of H₂O and 160 microliter of ethylene glycol was added to a 45 mL Teflon-insert autoclave (Model 4744 General Purpose Acid Digestion Vessel, Parr). While stirring, 2.7126 g of MoO₃, 0.5141 g of V₂O₅, 0.4373 g of Bi₂O₃, 0.6389 g of Pr₆O₃, 0.286 g of Nb₂O₅·xH₂O, 0.5422 g of Citric acid and 0.2388 g of oxalic acid was added sequentially and stirred for 10 min. Hydrothermal synthesis of MoV_{0.3}Nb_{0.1}Bi_{0.1}Pr_{0.2}Oₓ was performed in a rotating shaft oven at 190° C. for 48 hours rotating at 10 rpm. The obtained material from the hydrothermal synthesis was purified with 90 mL of deionized water using vacuum filtration and subsequently dried at 85° C. overnight.

After drying, the material was calcined at 450° C. (at a heating rate of 2° C./min) under a flow of N₂, for 2 hours. The material was compacted under 7 ton pressure and crushed and sieved to 40-80 mesh prior to loading in the reactor and tested at 1.25 bar(a) ethane pressure having a WHSV of 3.2 hr⁻¹.

### Performance Testing

Performance testing was performed in a fixed-bed reactor set-up. For catalytic testing, the appropriate amount of 40-80 mesh catalyst particles were loaded in the reactors, and the reactors were operated in cyclic mode in which periods of ethane exposure are alternated with oxidative regeneration at the desired temperature:
LODh step a feed stream comprising 50 vol % ethane in He/N₂ was used. The partial pressure of ethane (Pₑₜₕₐₙₑ) was 1.25 bar(a) to 2.5 bar(a), a WHSV of 2.3/hr to 3.2/hr was used.
Regeneration step conducted reoxidation in diluted (2.5 vol % O₂) air at a pressure of 2.5 bar(a) to 5 bar(a).

The reactor effluent composition was obtained by gas chromatography (GC) and the conversion and carbon based selectivities are calculated using the following equations:

$${X C}_{2} {H}_{6} ( \% ) = [ ( η {C}_{2} {H}_{6} , in - η {C}_{2} {H}_{6} , out ) / η {C}_{2} {H}_{6} , in ] \cdot 100 ; and \qquad ( 1 )$$

$${S}_{j} ( \% ) = [ α j \cdot η j , out / Σα j \cdot η j , out ] \cdot 100 \qquad ( 2 )$$

where XC₂H₆ is defined as the C₂H₆ conversion (%), η, in is defined as the molar inlet flow of the component (mol/min), η, out is the molar outlet flow of the component (mol/min), Sⱼ is defined as the carbon based selectivity to product j (%), αj the number of carbon atoms for product j. Carbon balance for all experiments was within 99-102% for all experiments.

The catalyst/ethane ratio (g/g) is calculated based on the time-on-stream (TOS, min) in which the GC analyzes the reactor effluent:

$$Cat / ethane = w / ( TOS \cdot η {C}_{2} {H}_{6} , in \cdot {MW}_{C 2 H 6} ) \qquad ( 3 )$$

where w is defined as the catalyst mass, ηC₂M₆, in is the molar inlet flow of ethane (mol/min) and MW_{C2H6} is the molecular weight of ethane (30 g/mol).

TABLE 2
Catalytic Performance of selected examples in anaerobic lattice oxidative
dehydrogenation of Ethane at 450° C. and 1.25 bar(a) of ethane

| | | | | Yield | | | | |
|---|---|---|---|---|---|---|---|---|
| | | XC₂H₆ | SC₂H₄ | C₂H₄ | SCO | SCO₂ | SCH₄ | |
| | Cycle | (%) | (%) | (%) | (%) | (%) | (%) | Cat/Ethane |
| Example 1 | 2-5 | 20.7 | 71.0 | 14.7 | 14.1 | 14.7 | 0.2 | 110 |
| | 10-15 | 24.0 | 81.7 | 19.6 | 9.8 | 8.5 | <0.1 | 110 |
| | 20-25 | 22.9 | 84.9 | 19.4 | 8.3 | 6.8 | <0.1 | 110 |
| | 30-35 | 20.5 | 85.6 | 17.5 | 7.9 | 6.5 | <0.1 | 110 |
| Example 2 | 2-5 | 24 | 86.5 | 20.8 | 7.4 | 6.1 | <0.1 | 95 |
| | 10-15 | 26.2 | 87.6 | 23.0 | 6.9 | 5.5 | <0.1 | 95 |
| | 20-25 | 27.6 | 87.8 | 24.2 | 6.8 | 5.4 | <0.1 | 95 |
| Example 3 | 2-5 | 13.3 | 70.1 | 9.3 | 16.8 | 12.2 | <0.1 | 77 |
| | 10-15 | 11.5 | 73.3 | 8.4 | 15.9 | 10.8 | <0.1 | 77 |
| | 20-25 | 10.4 | 75.4 | 7.8 | 14.9 | 9.7 | <0.1 | 77 |
| | 30-35 | 11 | 76.1 | 8.4 | 14.5 | 9.4 | 0 | 77 |
| Example 4 | 2-5 | 15.5 | 64.7 | 10.0 | 17.4 | 17.9 | <0.1 | 78 |
| | 10-15 | 17.4 | 67 | 11.7 | 19.2 | 13.8 | <0.1 | 78 |
| | 20-25 | 17.4 | 68.5 | 11.9 | 19.4 | 12.1 | <0.1 | 78 |
| Example 5 | 2-5 | 19.1 | 82.6 | 15.8 | 9.9 | 7.5 | <0.1 | 98 |
| | 10-15 | 18.2 | 83.6 | 15.2 | 9.4 | 7.1 | <0.1 | 98 |
| Comp. Ex 1 | 2-4 | 24 | 89 | 21.4 | 6 | 5 | <0.1 | 61 |
| | 29-32 | 14 | 92 | 12.9 | 5 | 3 | <0.1 | 61 |

### Example 6

Example 6 utilizes the same catalyst as Example 2, but was tested at 425° C. having a WHSV of 2.3/hr and an ethane partial pressure of 2.5 bar(a).

### Example 7

A mixture of 34 mL of H₂O and 160 microliter of ethylene glycol was added to a 45 mL Teflon-insert autoclave (Model 4744 General Purpose Acid Digestion Vessel, Parr). While stirring, 2.7126 g of MoO₃, 0.5141 g of V₂O₅, 0.2186 g of Bi₂O₃, 0.1517 g of Sb₂O₅, 0.286 g of Nb₂O₅·xH₂O, 0.5422 g of Citric acid and 0.2388 g of oxalic acid was added sequentially and stirred for 10 min. Hydrothermal synthesis of MoV_{0.3}Nb_{0.1}Sb_{0.05}Bi_{0.05}Oₓ was performed in a rotating shaft oven at 190° C. for 48 hours rotating at 10 rpm. The obtained material from the hydrothermal synthesis was purified with 90 mL of deionized water using vacuum filtration and subsequently dried at 85° C. overnight.

After drying, the material was calcined at 450° C. (at a heating rate of 2° C./min) under a flow of N₂, for 2 hours. The material was compacted under 7 ton pressure and crushed and sieved to 40-80 mesh prior to loading in the reactor and tested at 450° C., 2.5 bar(a) ethane pressure having a WHSV of 3.2/h.

TABLE 3
Catalytic Performance of selected examples in anaerobic
lattice oxidative dehydrogenation of Ethane

| | | XC₂H₆ | SC₂H₄ | Yield | SCO | SCO₂ | |
|---|---|---|---|---|---|---|---|
| | Cycle | (%) | (%) | C₂H₄ (%) | (%) | (%) | Cat/Ethane |
| Example 6 | 33-37 | 27.5 | 85.6 | 23.5 | 6.9 | 7.4 | 103 |
| | 38-42 | 29.9 | 85.8 | 25.7 | 7 | 7.2 | 103 |
| Example 7 | 15-20 | 21.3 | 81.3 | 17.3 | 10.2 | 8.5 | 113 |
| | 45-50 | 22.0 | 80.9 | 17.8 | 10.3 | 8.7 | 113 |

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A method for converting alkanes to olefins comprising:
contacting a feed stream comprising alkanes with an oxidative dehydrogenation catalyst in a reaction zone, where the oxidative dehydrogenation catalyst does not comprise tellurium; and
dehydrogenating the alkanes in the reaction zone without a co-feed of oxygen to yield a product stream comprising olefins, wherein
the oxidative dehydrogenation catalyst has the following formula:
MoᵥV_{w}Nb_{y}A_{z}Oₓ, where
v is 1.0,
w is from 0.1 to 0.5,
y is from 0.001 to 0.3,
A is Bi, Sb, Pr, or mixtures thereof,
z is from 0.01 to 0.3, and
x is an oxygen content required to charge-balance the structure, and
the oxidative dehydrogenation catalyst has a crystallographic structure with Pba2-32 space group, characterized by reflections determined with Cu-K_{α} X-ray diffraction (XRD) as follows:

| 2θ (±0.3°) | Rel. Intensity (%) |
|---|---|
| 5.3 | 0.2-8 |
| 6.6 | 1.5-15 |
| 7.84 | 2.5-45 |
| 8.95 | 4-21 |
| 22.17 | 100 |
| 27.2 | 20-50 |
| 28.1 | 10-30. |

2. A method for converting alkanes to olefins comprising:
contacting a feed stream comprising alkanes with an oxidative dehydrogenation catalyst in a reaction zone, where the oxidative dehydrogenation catalyst has the following formula:
MoᵥV_{w}Nb_{y}Bi_{z}Oₓ, where
v is 1.0,
w is from 0.1 to 0.5,
y is from 0.001 to 0.3,
z is from 0.01 to 0.3, and
x is an oxygen content required to charge-balance the structure,
wherein the oxidative dehydration catalyst has a crystallographic structure with Pba2-32 space group, characterized by reflections determined with Cu-K_{α} X-ray diffraction (XRD) as follows:

| 2θ (±0.3°) | Rel. Intensity (%) |
|---|---|
| 5.3 | 0.2-8 |
| 6.6 | 1.5-15 |
| 7.84 | 2.5-45 |
| 8.95 | 4-21 |
| 22.17 | 100 |
| 27.2 | 20-50 |
| 28.1 | 10-30; |

and
dehydrogenating the alkanes in the reaction zone to yield a product stream comprising olefins.

3. The method of claim 2, wherein the dehydrogenation occurs in the presence of molecular oxygen.

4. The method of claim 1, wherein the dehydrogenation occurs without a presence of oxygen.

5. The method of claim 1, wherein the dehydrogenating comprises contacting the feed stream with the oxidative dehydration catalyst in the reaction zone at a temperature from 300° C. to 700° C.

6. The method of claim 1, wherein the dehydrogenating comprises contacting the feed stream with the oxidative dehydration catalyst in the reaction zone at a temperature from 400° C. to 500° C.

7. The method of claim 1, wherein the dehydrogenating comprises contacting the feed stream with the oxidative dehydration catalyst in the reaction zone at a pressure from 0 bar(g) (0 KPa) to 20 bar(g) (2000 KPa).

8. The method of claim 1, wherein the dehydrogenating comprises contacting the feed stream with the oxidative dehydration catalyst in the reaction zone at a pressure from 0 bar(g) (0 KPa) to 10 bar(g) (1000 KPa).

9. The method of claim 1, wherein the dehydrogenating comprises contacting the feed stream with the oxidative dehydration catalyst in the reaction zone where the feed stream has a weight hour space velocity (WHSV) from 1/hr to 10/hr.

10. The method of claim 1, wherein
the reaction zone is selected from the group consisting of a fluidized bed reactor, a moving bed reactor, a fixed bed reactor, a reverse flow reactor, or an ebullated bed reactor.

11. The method of claim 10, wherein the reaction zone is a fluidized bed reactor.

12. The method of claim 10, wherein the oxidative dehydration catalyst is regenerated in the regeneration zone using an oxygen-containing gas stream having from 2 vol % to 22 vol % oxygen.

13. The method of claim 12, wherein the oxygen-containing gas stream is diluted or undiluted air.

14. The method of claim 11, wherein a pressure in the regeneration zone is from 0 bar(g) (100 KPa) to 21 bar(g) (1000 KPa).

15. The method of claim 1, wherein the product stream is further processed to remove at least one of oxygenates, carbon monoxide, carbon dioxide, and alkanes from the product stream.

16. The method of claim 2, wherein the dehydrogenation occurs without a presence of oxygen.

17. The method of claim 2, wherein the dehydrogenating comprises contacting the feed stream with the oxidative dehydration catalyst in the reaction zone at a temperature from 300° C. to 700° C.

18. The method of claim 2, wherein the dehydrogenating comprises contacting the feed stream with the oxidative dehydration catalyst in the reaction zone at a pressure from 0 bar(g) (0 KPa) to 20 bar(g) (2000 KPa).

19. The method of claim 2, wherein the dehydrogenating comprises contacting the feed stream with the oxidative dehydration catalyst in the reaction zone where the feed stream has a weight hour space velocity (WHSV) from 1/hr to 10/hr.

20. The method of claim 2, wherein
the reaction zone is selected from the group consisting of a fluidized bed reactor, a moving bed reactor, a fixed bed reactor, a reverse flow reactor, or an ebullated bed reactor.
